(19) United States
(12) Patent Application Publication
(10) Pub. No.: US 2017/0369574 A1
(43) Pub. Date: Dec. 28, 2017

(54) AN IMMUNOTOXIN FOR USE IN THE TREATMENT OF LEISHMANIASIS

(71) Applicant: FRAUNHOFER-GESELLSCHAFT ZUR FÖRDERUNG DER ANGEWANDTEN FORSCHUNG E.V., Munich (DE)

(72) Inventors: Stefan Barth, Aachen (DE); Theophilus Thepen, Aachen (DE); Manoel Barral-Netto, Lauro De Freitas (BR); Aldina Barral, Lauro De Freitas (BR); Johan Van Weyenbergh, Heverlee (BE)

(73) Assignee: FRAUNHOFER-GESELLSCHAFT ZUR FÖRDERUNG DER ANGEWANDTEN FORSCHUNG E.V., Munich (DE)

(21) Appl. No.: 15/542,549

(22) PCT Filed: Jan. 21, 2016
(86) PCT No.: PCT/EP2016/051208

§ 371 Date: Jul. 10, 2017

(30) Foreign Application Priority Data
Jan. 21, 2015 (EP) 15151876.8

(51) Int. Cl. C07K 16/28; C12N 9/24; C12N 9/10; A61K 39/00

(52) U.S. Cl. CPC C07K 16/283; C12Y 302/02022; C12N 9/1077; C12N 9/2497; C12Y 204/02036; C07K 2317/622; C07K 2319/01; C07K 2319/10; C07K 2319/55; A61K 2039/505; C07K 2319/74

(57) ABSTRACT

An immunotoxin for use in the treatment of leishmaniasis A wherein the immunotoxin comprises a portion which is specifically binding to the cellular surface receptor CD64 as a component A and a cell killing portion as a component B, wherein the cell killing portion alters the function, gene expression, or viability of a cell thereby killing Leishmania-infected macrophages and by this eliminates Leishmania.

The present invention pertains to an immunotoxin for use in the treatment of leishmaniasis.

Leishmaniasis is a neglected infectious disease, despite the 1.5-2.0 million new cases every year and a population of 350 million at risk [1]-[3]. The causative agent is a protozoan parasite from the genus Leishmania, transmitted by bites from infected phlebotomine sand flies. Cutaneous leishmaniasis (CL) is an emerging infectious disease in several countries, due to behavioural and environmental changes, as well as malnutrition and HIV co-infection [2]. Due to worldwide increasing therapeutic failure and drug resistance, as well as serious side effects of current chemotherapy, new drugs are urgently needed for CL [4]. In addition, no surrogate markers are available to monitor clinical response during often lengthy periods of treatment or to predict therapeutic failure.

J. Van Weyenbergh et al. discloses in Abstract/Cytokine 63 (2013) 243-314 that CD64-targeted immunotoxins selectively induce apoptosis and decrease parasite survival in Leishmania-infected human macrophages in vitro. CD64-immunotoxin treatment in vivo decreases lesion size, parasite load and inflammation in infected HuCD64-transgenic mice, but not control WT mice. The authors conclude that the results reveal the therapeutic potential of CD64-immunotoxin treatment in cutaneous leishmaniasis, but also challenge the clinical relevance of Thl boosting, as suggested in established murine models, for therapeutic and vaccination strategies in human leishmaniasis.

WO 2005/052007 A1 discloses a heterologous, recombinantly prepared complex which comprises at least one cytotoxic domain and at least one CD64-specific binding domain, especially of human origin, and nucleic acids and vectors coding for such a complex. It further reports about methods for influencing cell growth and the physiology of CD64-positive cells with the complex according to the invention or with vectors containing the nucleic acid coding therefore. Further vectors and hosts for producing the complex are disclosed. The disclosure further relates to the preparation and distribution of medicaments based on the complex or vectors coding therefore, for the treatment of diseases based on a pathological proliferation and/or increased activity of structurally defined cell populations. This applies, in particular, to tumour diseases, allergies, autoimmune diseases, infectious diseases, chronic inflammation or transplantations (immune suppression).

An object of the present invention is to provide a new therapeutic approach for treating leishmaniasis.

The present invention is based on the observation that monocyte CD64 (FcγRI) ex vivo expression is elevated in two independent cohorts of cutaneous leishmaniasis patients and significantly predicts therapeutic failure. CD64-targeted immunotoxins composed of a receptor-specific antibody combined with a cell killing portion selectively induce apoptosis and decrease parasite survival in Leishmania-infected human macrophages. In vivo immunotoxin treatment of infected HuCD64-transgenic mice decreases lesion size, parasite load and inflammation, providing proof-of-concept for targeting the pathogen by eliminating the host cell in protozoan infection. The invention was verified by means of the CD64-targeting immunotoxin H22xRA composed of the H22 full length antibody chemically conjugated to the plant ribosome-inactivating protein ricin A and H22-ETA a recombinant fusion protein of H22 single-chain antibody fragment genetically fused to a mutant of Pseudomonas exotoxin A. However to avoid any undue limitation, the present invention as described herein is of course not limited to the use of that specific molecule. It must be clear that the skilled person understands, that once the mechanism underlying the therapeutic approach provided by the present invention is elucidated, a plethora of potential molecules can be designed which are able to treat successfully leichmaniasis.

Previously, it was shown in a Tuberculosis infection model in mice, that specific in vitro elimination of mycobacteria infected Alveolar Macrophages with H22xRA immunotoxin did not lead to pathogen elimination, but rather to exacerbation of the disease, followed by death of the mice. This indicates that for Leishmania infection, the H22 based immunotoxin provides additional selectivity resulting in bringing the parasite laden monocytic cells into apoptosis, concurrently killing the intracellular parasites, without disturbing the abiding immune response, fighting the Leishmania parasite [5] incorporated by reference.

The object underlying the invention is accomplished by an immunotoxin for use in the treatment of leishmaniasis wherein the immunotoxin comprises a portion which is specifically binding to the cellular surface receptor CD64 as a component A and a cell killing portion as a component B, wherein the cell killing portion alters the function, gene expression, or viability of a cell thereby killing Leishmania-infected macrophages and by this eliminates Leishmania.

Necessary is the component A which binds to the CD64 receptor molecule which expression in macrophages is elevated, which are infected with Leishmania. This component must have an affinity to the CD64 molecule.

Furthermore a component B must be present which is able to interact after uptake by the infected cell, i.e a macrophage, with that cell and kill that cell either non specifically or by an induced cell-death. The present invention therefore provides an immunotoxin for use in the treatment of leishmaniasis wherein the immunotoxin comprises a portion, which is specifically binding to the cellular surface receptor CD64, and a cell killing portion.

In particular the immunotoxin of the invention comprises a cell killing portion which is covalently bonded to the portion specifically binding to the cellular surface receptor CD64.

In one embodiment of the invention the immunotoxin is a recombinant protein or the portion specifically binding to the cellular surface receptor CD64 is linked directly to the cell-killing portion or linked via a linking group.

In a further embodiment of the invention the portion of the immunotoxin which is specifically binding to the cellular surface receptor CD64 is selected from the group consisting of antibodies or their derivatives or fragments, synthetic peptides or molecules, ligands, receptor binding molecules, and their structural analogs, mutants or combinations thereof.

The term “mutant” as used herein is well understood by the person skilled in the art. Mutants are basically proteins which are the product of mutated genes, i. e. proteins which have an altered sequence of amino acids in comparison with the wild type. The result of an amino acid exchange in the mutant may be substantially neutral i.e. without effects on the function, for example if a conservative exchange of amino acids occurs. Mutant in the sense of the present invention comprises also activating mutations, change the gene product such that its effect gets stronger (enhanced activation). Methods for obtaining and identifying mutants are well known in the art, such as site directed mutagenesis in particular in combination with high-trough-put screening (HTS).

The skilled person knows the term “structural analog” as chemical compound that can be imagined to arise from another compound, if one atom or group of atoms is replaced with another atom or group of atoms. According to the invention the structural analog shall have substantially the same activity as the compound from which it has been derived.

The term “small molecule” is understood as a low molecular weight organic compound, typically involved in a biological process as a substrate or product typically in within a mass range of 50-1500 daltons (Da). Some examples of small molecules include: sugars, lipids, amino acids, fatty acids, phenolic compounds, alkaloids etc.

In still another embodiment of the invention the portion, which is specifically binding to the cellular surface receptor CD64, is a recombinant molecule.

In yet another embodiment of the invention the cell killing portion alters the function, gene expression, or viability of a cell in particular by inactivating molecules responsible for protein biosynthesis or activating components of cell-inherent apoptosis pathways.

In a further embodiment of the invention the cell killing portion is cytotoxic in particular a molecule selected from the group consisting of a member of ADP-ribosylating enzymes, such as the Pseudomonas Exotoxin A, Diphtheria-, Cholera- or the Pertussis-, Botulinumtoxin; or a member of the ribosome-inactivating proteins such as Dianthin, Saporin, Bryodin, Gelonin, Ricin, Abrin, Pokeweed Antiviral Protein (PAP) or Restrictocin; or is a member of the RNases (Phosphodiesterases) such as the Bovine seminal RNase, BovineRNase A, Bovine pancreatic RNase, Angiogenin, Eosinophil-derived Neurotoxin (EDN), Eosinophilic Cationic Protein (ECP), Onconase, or Bullfrog Lectin; or is a member of the prodrug-activating enzymes such as Calicheamicin, Glucose Oxidase, Carboxypeptidase, Alkaline Phosphatase, Cytosindeaminase, β-Glucosidase, β-Glucuronidase, β-Lactamase, Nitroreductase, Thymidinkinase or Purin Nukleosid Phosphorylase; or is a member of the cathepsin protease family; or a member of the calpains; or a member of the granzymes; or a member of the microtubule-binding proteins including tau; or any derivative of the above mentioned proteins; or a combination thereof. The term “derivative” in the context of proteins means covalently modified proteins, in particular amidated, alkylated, phosphorylated, acylated, glycosylated, etc. N- or C-terminally or side chain modified proteins.

In particular the cell killing portion of the immunotoxin of the invention include all kinds of substances that can display proven efficacy e.g. as chemotherapeutical agents and may be selected from the group of alkylating agents (e.g. cyclophosphamide, cholrambucil), anthracyclins (doxorubicin, daunomycin), maytansinoids (maytansinoid DM1), anti-metabolites, plant alkaloids and terpenoids as the Vinca alkaloids (vinblastine, vincristine vinorebline, vindesin) Podophyllotoxin and structural analogs hereof and taxanes (paclitaxel, docetaxel, taxotere) or topoisomerase inhibitors (camptothecins), synthetic toxins as ellipticine analogs or synthetic analogs of tumor antibiotics as duocarmycin or CC1065, other tubulin binding agents as halichondrin B, hemiasterlins and dolastatins or analogs as monomethyl-auristatin E; component B may also be selected from the group of small molecules having cytotoxic/cytostatic activities like alkylating agents (like Cyclophosphamide, Mechlorethamine, Chlorambucil, Melphalan) or anthracyclines (like Danorubicin, Doxorubicin, Epirubicin, Idarubicin, Mitoxantrone, Valrubicin) or cytoskeletal disruptors (like Paclitaxel, Docetaxel) or Epothilones (like) or Inhibitors of topoisomerase II (like Etoposide, Teniposide, Tafluposide) or nucleotide analogs and precursor analogs (like azacididine, azathioprine, capecitabine, cytarabine, doxofluridine, fluorouracil, gemcitabine, mercaptopurine, methotrexate, tioguanine) or peptide antibiotics (like bleomycin) or platinum-based agents (like carboplatin, cisplatin, oxaliplatin) or retinoids (like all-trans retinoic acid) or vinca alkaloids and structural analogs (like vinblastine, vincristine, vindestine, vinorelbine), beta ray emitting nuclides like Iodine-131, Yttrium-90, Lutetium-177, from the group of Aromatase Inhibitors (like Aminoglutethimide, Anastrozole, Letrozole, Vorozole, Exemestane, 4-androstene-3,6,17-trione, 1,4,6-androstatrien-3,17-dione, Formestane, Testolactone), Carbonic Anhydrase Inhibitors (like Acetazolamide, Methazolamide, Dorzolamide, Topiramate), Cholinesterase Inhibitors (Organophosphates like Metrifonate, Carbamates like Physostigmine, Neostigmine, Pyridostigmine, Ambenonium, Demarcarium, Rivastigmine, Phananthrine like Galantamine, Piperidine like Donepezil, Tacrine, Edophonium, or Phenothiazines), Cyclooxygenase Inhibitors (like Celecoxib, Rofecoxib, Etoricoxib, Acetaminophen, Diclofenac, Ibuprofen), Folic Acid Antagonists (like Methotrexate), Hydroxymethylglutaryl-CoA Reductase Inhibitors (like Atorvastatin, Cerivastatin, Fluvastatin, Lovastatin, Mevastatin, Pitavastatin, Pravastatin, Rosuvastatin, Simvastatin, Vytorin, Advicor, Caduet), Integrase Inhibitors (like Raltegravir, Elvitegravir), Lipoxygenase Inhibitors (like Zileutron), Monoamine Oxidase Inhibitors (like Isocarboxazid, Moclobemide, Phenelzine, Tranylcypromine, Selegiline, Rasagiline, Nialamide, Iproniazid, Iproclozide, Toloxatone, Linezolid, Tryptamines, Dienolide, Detxtroamphetamine), Nucleic Acid Synthesis Inhibitors, Phosphodiesterase Inhibitors (like Caffeine, Theopyline, 3-isobutyl-1-methylxanthine, Vinpocetine, EHNA, Enoximone, Lirinone, PDE3, Mesembrine, Rolipram, Ibudilast, Sildenafil, Tadalafil, Vardenafil, Udenafil, Avanafil), Protease Inhibitors (like Saquinavir, Ritonavir, Idinavir, Nelfinavir, Amprenavir, Lopinavir, Atazanavir, Fosamprenavir, Tipranavir, Darunavir), Protein Kinase Inhibitors (like Imatinib, Geftinib, Pegaptanib, Sorafenib, Dasatinib, Sunitinib, Erlotinib, Nilotinib, Lapatinib), Protein Synthesis Inhibitors (like Anisomycin, Cycloheximide, Chloramphenicol, Tetracycline, Streptomycin, Erythromycin, Puromycin, etc.), Proton Pump Inhibitors (like Omeprazole, Lansoprazole, Esomeprazole, Pantoprazole, Rabeprazole), from the group of oligonucleotides nucleic acids like small interfering RNAs (siRNAs) or a short hairpin RNA (shRNA), an antisense DNA or RNA, a double stranded RNA (dsRNA) or a micro RNA (miRNA) might be used to down-regulate specific key elements of regulative pathways within a cell.

In a particular embodiment of the immunotoxin of the invention at least one supplementary component C is additionally present. Preferably the component C regulates expression of a gene encoding the complex. It may also be used for purification of the recombinant complex or its individual components alone as A or B, or of the components together as AB. It may also contain structural subcomponents which stimulate internalization of the immunotoxin or its individual components, in particular of the cell killing portion, into a macrophage as target cell. The component C may also trigger translocation of the cell killing portion into a subcellular compartment or stimulate proteolytic removal of the portion which is specifically binding to the cellular surface receptor CD64 from the cell killing portion or trigger intracellular activation of the cell killing portion. The skilled person readily understands that component C may possess only a single one of the features as described hereinabove or combinations thereof.

In a further embodiment of the invention the cell killing portion is cytotoxic in particular a molecule selected from the group consisting of a member of ADP-ribosylating enzymes, such as the Pseudomonas Exotoxin A, Diphtheria-, Cholera- or the Pertussis-, Botulinumtoxin; or a member of the ribosome-inactivating proteins such as Dianthin, Saporin, Bryodin, Gelonin, Ricin, Abrin, Pokeweed Antiviral Protein (PAP) or Restrictocin; or is a member of the RNases (Phosphodiesterases) such as the Bovine seminal RNase, BovineRNase A, Bovine pancreatic RNase, Angiogenin, Eosinophil-derived Neurotoxin (EDN), Eosinophilic Cationic Protein (ECP), Onconase, or Bullfrog Lectin; or is a member of the prodrug-activating enzymes such as Calicheamicin, Glucose Oxidase, Carboxypeptidase, Alkaline Phosphatase, Cytosindeaminase, β-Glucosidase, β-Glucuronidase, β-Lactamase, Nitroreductase, Thymidinkinase or Purin Nukleosid Phosphorylase; or is a member of the cathepsin protease family; or a member of the calpains; or a member of the granzymes; or a member of the microtubule-binding proteins including tau; or any derivative of the above mentioned proteins; or a combination thereof.

A single-chain variable fragment (scFv) is a fusion protein of the variable regions of the heavy (V_{H}) and light chains (V_{L}) of immunoglobulins, connected with a short linker peptide of ten to about 25 amino acids. The linker is usually rich in glycine for flexibility, as well as serine or threonine for solubility, and can either connect the N-terminus of the V_{H} with the C-terminus of the V_{L}, or vice versa. This protein retains the specificity of the original immunoglobulin, despite removal of the constant regions and the introduction of the linker. These molecules were created to facilitate phage display, where it is highly convenient to express the antigen-binding domain as a single peptide. As an alternative, scFv can be created directly from subcloned heavy and light chains derived from a hybridoma. ScFvs have many uses, e.g., flow cytometry, immunohistochemistry, and as antigen-binding domains of artificial T cell receptors. Unlike monoclonal antibodies, which are often produced in mammalian cell cultures, scFvs are more often produced in bacteria cell cultures such as E. coli. [6]

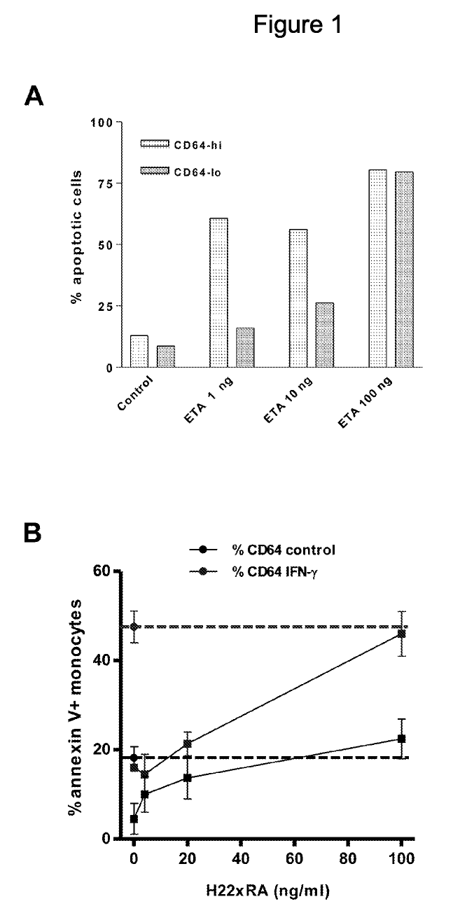

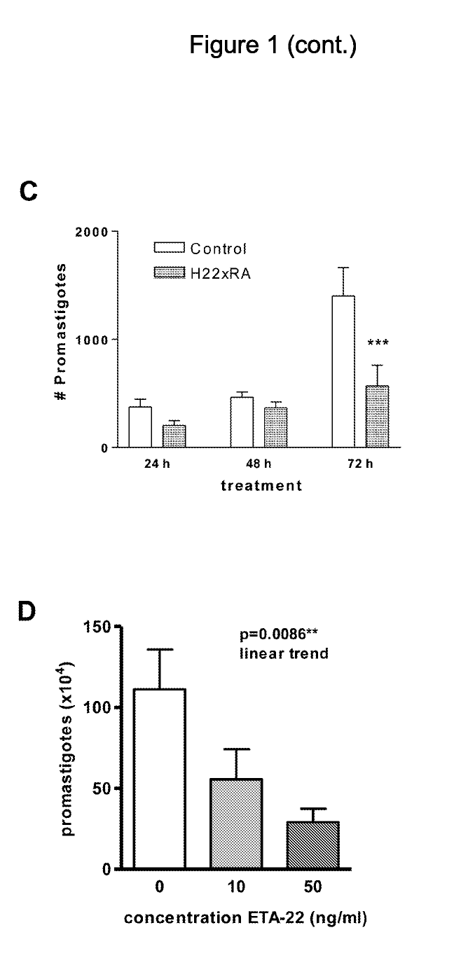

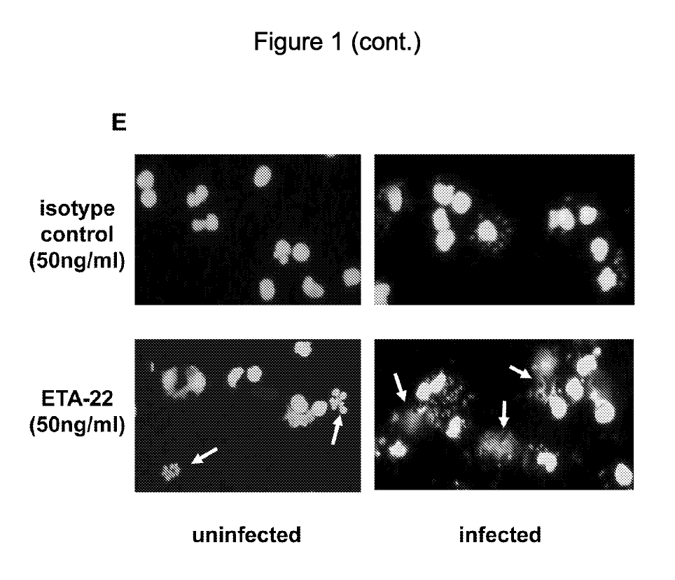

FIG. 1a-e show CD64-directed immunotoxins induce apoptosis in a CD64-selective manner and exert leishmanicidal activity in vitro.

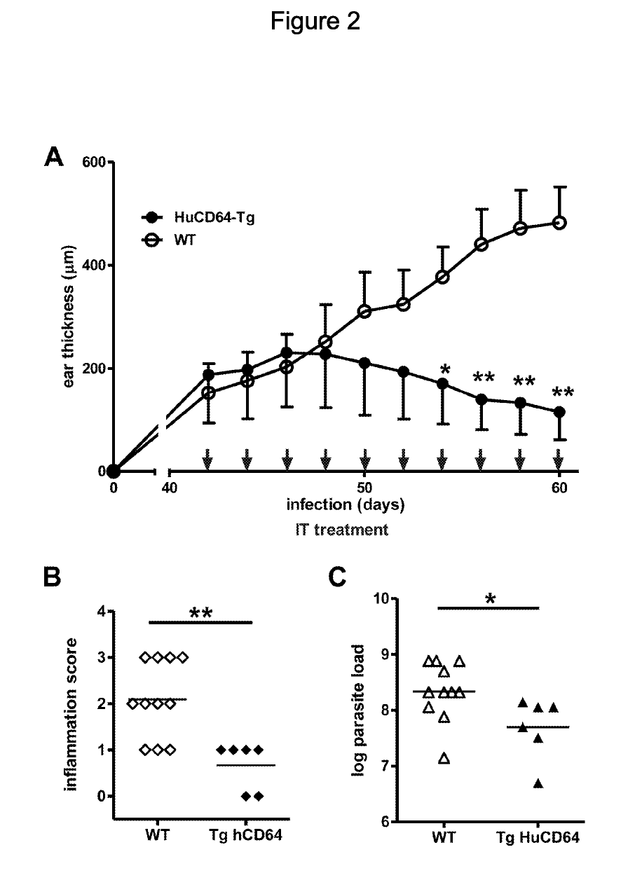

FIG. 2a-c show CD64-directed immunotoxin demonstrates therapeutic potential in vivo.

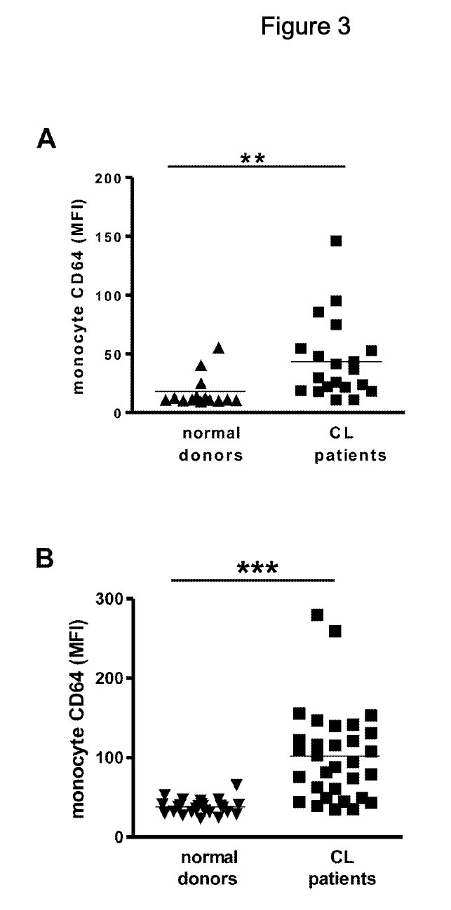

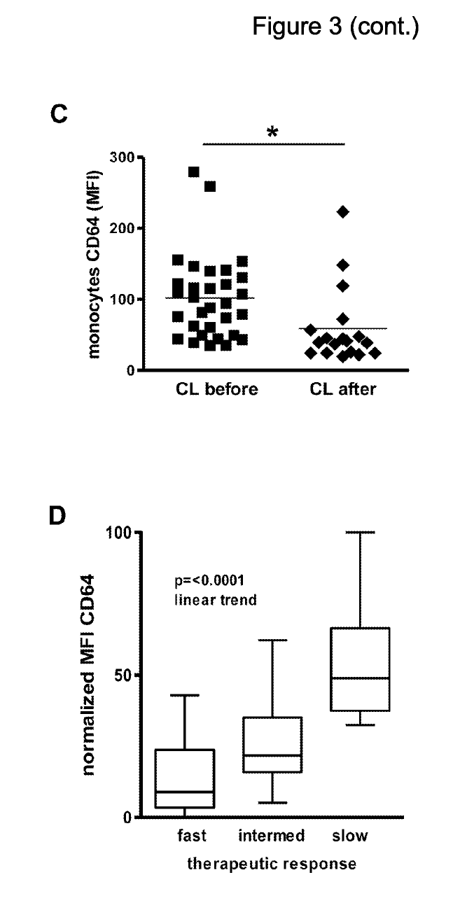

FIG. 3a-d show CD64 is a biomarker for therapeutic response in cutaneous leishmaniasis.

### DETAILED DESCRIPTION OF THE INVENTION

Bacterial Strains, Mammalian Cells, and Plasmids

E. coli BL21 Derivatives including (DE3) (F⁻ ompT hsdS_{B}(r_{B}⁻m_{B}⁻) gal dcm rne131 DE3) were as host for bacterial synthesis of ETA′-, Ang-, and tau-based fusion proteins. The bacterial expression vector pBM1.1 is derived from the pET27b plasmid (Novagen, Madison, USA), and is used for N-terminal fusion of Sfi I/Not I-ligands to the modified deletion mutant of Pseudomonas aeruginosa Exotoxin A plasmids were prepared by the alkaline lysis method and purified using plasmid preparation kits from Qiagen (Hilden, Germany) [7], incorporated by reference. All standard cloning procedures were carried out as described by HETK293T cells were used as host for expression of GB, GM, and Ang-based fusion proteins [8], incorporated by reference. The construction of pMS plasmids encoding GB-H22 has already been described [9], incorporated by reference.

Construction and Expression of CD64-Specific Recombinant Immunotoxins

After transformation into BL21 (DE3) strains, H22(scFv)-ETA′ (SEQ ID NO: 1), H22(scFv)-Ang (SEQ ID NO: 2), H22(scFv)-_{Cat}AD-Ang (SEQ ID NO: 3), H22(scFv)-Ang_{GGRR} (SEQ ID NO: 4), H22(scFv)-_{Cat}AD-Ang_{GGRR} (SEQ ID NO: 5), H22(scFv)-_{Cat}AD-GB (SEQ ID NO: 6), H22(scFv)-_{Cat}AD-GB_{R201K} (SEQ ID NO: 7), H22(scFv)-MAP (SEQ ID NO: 8), fusion proteins were periplasmically expressed under osmotic stress in the presence of compatible solutes as described by Barth et al. 2000. Briefly, transformed bacteria were harvested 15 h after IPTG induction. The bacterial pellet was resuspended in sonication-buffer (75 mM Tris/HCl (pH 8), 300 mM NaCl, 1 capsule of protease inhibitors/50 ml (Complete™, Roche Diagnostics, Mannheim, Germany), 5 mM DTT, 10 mM EDTA, 10% (v/v) glycerol) at 4° and sonicated 6 times for 30 s at 200 W. The m22(scFv)-ETA′ fusion proteins were enriched by IMAC (immobilized metal-ion affinity chromatography) using nickel-nitriloacetic chelating Sepharose (Qiagen) and SEC (size exclusion chromatography) with Bio-Prep SE-100/17 (Biorad, München, Germany) columns according to the manufacturer's instructions. Recombinant Protein was eluted with PBS (pH 7.4) and 1 M NaCl, analyzed by Sodium dodecyl sulfate/polyacrylamide gel electrophoresis (SDS-PAGE), quantified by densitometry (GS-700 Imaging Densitometer; Biorad) after Coomassie staining in comparison with BSA standards and verified by Bradford assays (Biorad).

HEK293T cells were used as expression cell line. The cells were transfected with 1 μg DNA, GB-H22(scFv) (SEQ ID NO: 9), GB_{R201K}-H22(scFv) (SEQ ID NO: 10), GM-H22(scFv) (SEQ ID NO: 11), H22(scFv)-_{Cat}AD-GB (SEQ ID NO: 6), H22(scFv)-_{Cat}AD-GB_{R201K} (SEQ ID NO: 7), H22(scFv)-Ang (SEQ ID NO: 2), H22-_{Cat}AD-Ang (SEQ ID NO: 3), H22(scFv)-Ang_{GGRR} (SEQ ID NO: 4), and H22(scFv)-_{Cat}AD-Ang_{GGRR} (SEQ ID NO: 5), according to the manufacturer's instructions using RotiFect (Roth). The used pMS plasmid contains the EGFP reporter gene so that expression of the corresponding protein could be verified by its green fluorescence via fluorescence microscopy.

The secreted protein was purified from the supernatant of the cells via Immobilized Metal-ion Affinity Chromatography (IMAC) and Fast Performance Liquid Chromatography (FPLC). The cleared supernatant was supplemented with 10 mM imidazole and loaded to an XK16/20 column (Amersham/GE Healthcare) containing 8 ml Sepharose 6 Fast Flow resin (Clontech/Takara). The used buffers such as incubation, washing and elution buffer were described before [10], incorporated by reference. The eluted protein was re-buffered into 20 mM Tris, pH 7.4, 50 mM NaCl, concentrated, aliquoted and stored at −80° C. For activation prior to use Enterokinase was added to the protein (0.02 U/μg) with 2 mM CaCl2 for 16 h incubation at 23° C. The protein concentration was calculated after SDS-PAGE analysis and Coomassie staining using AIDA Image Analyzer Software (Raytest Isotopenmessgeräte GmbH).

In Vitro Cytotoxic Activity

To characterize the cytotoxic activity of the recombinant anti-CD64 immunotoxins in vitro, growth inhibition of AML-derived cell lines HL-60, U937 or in vitro differentiated macrophages was documented by XTT-based colorimetric assay (see Table 1).

TABLE 1

| | Expression | | | Target | | |
|---|---|---|---|---|---|---|
| Lead | system | Yield | | cells | IC50/EC50 | |
| ETA′ | E. coli | 1 | mg/l | U937 | 186 | pM |
| | E. coli | 1-1.5 | mg/l | U937 | 140 | pM |
| | E. coli | 1 | mg/l | HL-60 | 157 | pM |
| | E. coli | not mentioned | | HL-60 | 100-900 | pM |
| | E. coli | * | | HL-60 | 22 pM-2.5 nM | |
| | E. coli | * | | hM1φ | 0.689-15.08 | nM |
| | E. coli | not mentioned | | hM1φ | 214 | pM |
| GB | HEK293T | ~1 | mg/l | U937 | 1.7-17 | nM |
| | HEK293T | 1-2 mg/l | | HL-60 | 4-7 | nM |
| | HEK293T | not mentioned | | HL-60 | 0.2-2 | nM |
| | HEK293T | 1.5 | mg/l | hM1φ | 414 | pM |
| | HEK293T | 1-2 | mg/l | hM1φ | 140 | pM |
| CatAd-Gb | HEK293T | not mentioned | | HL-60 | 60-330 | pM |
| GBmut | HEK293T | not mentioned | | HL-60 | 4-7 | nM |
| | HEK293T | 1-2 | mg/l | HL-60 | 2.4 | nM |
| | HEK293T | 1.5 | mg/l | HL-60 | 5.7 | nM |
| | HEK293T | 4.9 | mg/l | HL-60 | 0.34-1.13 | nM |
| | HEK293T | 4.9 | mg/l | hM1φ | 0.18-0.43 | nM |
| CatAd- | HEK293T | ~0.4 | mg/l ** | HL-60 | 12-96.6 | pM |
| Gb_{R201K} | HEK293T | ~0.4 | mg/l ** | hM1φ | 56.9-64 | pM |
| GM | HEK293T | 1 | mg/l | HL-60 | 1.2-6.4 | nM |
| Ang | E. coli | ~1 | mg/l | U937 | 200 | pM |
| | HEK293T | not mentioned | | HL-59 | 2.8-5 | nM |
| | HEK293T | 1.7 | mg/l | HL-60 | 10 | nM |
| | HEK293T | 1.7 | mg/l | hM1φ | 290 | pM |
| | HEK293T | 1-2 mg/l | | hM1φ | 108 | pM |
| Ang_{GGRR} | HEK293T | 0.4-2.7 | mg/l | HL-60 | 6.7-0.57 | nM |
| | HEK293T | 0.4-2.7 | mg/l | hM1φ | 153-43 | pM |
| CatAD- | E. coli | ~1 | mg/l | U937 | 10 | pM |
| Ang | HEK293T | not mentioned | | HL-60 | 0.56-1.9 | nM |
| CatAD- | HEK293T | 0.5 | mg/l | HL-60 | 0.64 | nM |
| Ang_{GGRR} | HEK293T | 0.5 | mg/l | hM1φ | 0.79 | nM |
| MAP | E. coli | ~1 | mg/l | HL-60 | 40 | pM |

| | | | | |
|---|---|---|---|---|
| E. coli | ~1 | mg/l | hM1φ | unaffected |

Parasite Culture, Immunotoxin Preparation, and In Vitro Assays

Leishmania amazonensis (MHOM/BR/87/BA125) cultures were maintained in vitro as proliferating promastigotes in Schneider's insect medium (Sigma Chemical Co., St. Louis, Mo.), supplemented with 10% FCS at 25° C.

The construction and purification of CD64-directed immunotoxins H22xRA and H22-ETA single chain Fv have been described previously [10], [11], incorporated by reference. PBMCs were isolated by Ficoll-Hypaque gradient centrifugation. Monocytes were separated by adherence for 30 min, differentiated into macrophages for 7 days in RPMI+10% FCS (Gibco-BRL) and then infected with Leishmania amazonensis (5:1 ratio) before immunotoxin treatment (24-48 h). Intracellular parasite survival was quantified by transformation of amastigotes into motile promastigotes, which were allowed to proliferate in Schneider's medium for 8 days at 23° C. Apoptosis was assessed by nuclear fragmentation (Hoechst 33258 or hematoxyline/eosine staining, quantified by microscopy) and annexin V-staining (quantified by flow cytometry, FACSort, BD Biosciences).

Both immunotoxins were tested using Leishmania amazonensis-infected human macrophages from healthy donors, an established preclinical in vitro model. A significant time- and dose-dependent decrease in parasite survival was observed following treatment of infected macrophages with both H22xRA and H22-ETA (FIG. 1c-d). This immunotoxin-induced leishmanicidal activity was associated with host cell apoptosis, as shown in FIG. 1e. Control cultures exhibit characteristic large pleomorphic macrophage nuclei and small elongated Leishmania amastigote nuclei in infected cells (FIG. 1e). Upon immunotoxin treatment, both macrophage and Leishmania amastigote nuclei display nuclear fragmentation, structural degradation and loss of DNA content typical of apoptosis (arrows in FIG. 1e), indicating that host cell-targeting results in concurrent intracellular pathogen elimination.

Legend to FIG. 1:

CD64-directed immunotoxins induce apoptosis in a CD64-selective manner and exert leishmanicidal activity in vitro. a, In vitro apoptosis (quantified as % of nuclear fragmentation) of CD64 “high” (MFI>50) and “low” (MFI<50) monocytes from healthy donors cultured for 48 h in the presence or absence of 1, 10 or 100 ng of H22-ETA. b, In vitro apoptosis (quantified as % of annexin V-positive cells by flow cytometry) of untreated CD64 “low” and IFNγ-treated CD64 “high” monocytes from two healthy donors (mean±SEM) cultured for 48 h in the presence or absence of 1, 10 or 100 ng/ml of H22xRA. In vitro survival assay of Leishmania promastigotes recovered from Leishmania amazonensis-infected macrophages (duplicate cultures from four normal donors, mean±SEM) cultured for 24, 48 and 72 h in the presence or absence of c 100 ng/ml of H22-RA and d 10 and 50 ng/ml of H22-ETA. e, In vitro apoptosis visualized by Hoechst 33258 staining of uninfected and Leishmania amazonensis-infected macrophages from a representative healthy donor cultured for 48 h in the presence or absence of 50 ng/ml of H22-ETA immunotoxin (arrows indicate nuclear fragmentation and/or DNA degradation).

In Vivo Infection and Immunotoxin Treatment

Animal husbandry, experimentation and welfare complied with the International Guiding Principles for Biomedical Research Involving Animals and was approved by the Animal Care Ethics Committee from Uniklinikum Aachen. Human CD64-transgenic (described by Heijnen et al. [12], incorporated by reference) and WT C57BL6 mice were used at 8-12 weeks of age. Stationary-phase promastigotes (10⁵ parasites in 10 μl of saline) of Leishmania amazonensis were inoculated into the right ear dermis using a 27.5-gauge needle. At 6 weeks post-infection, both groups of mice were treated with 10 intralesional injections of 70 ng of H22-ETA (10 ul 1×10⁻⁷M in saline) on alternate days. Littermate WT mice were used as controls for non-specific effects of the immunotoxin, since murine CD64 is not recognized by the human mAb or immunotoxin. Lesion size was monitored every other day from 6 to 9 weeks post-infection using a digital micrometer (series 227/201 Mitutoyo Japan). Infected ears were aseptically excised at 9 weeks post-infection, photographed, scored for inflammation in a blinded manner by a trained pathologist and homogenized in Schneider's medium. Parasite load was determined using a quantitative limiting-dilution assay. Homogenates were serially diluted in Schneider's medium with 10% FCS and seeded into 96-well plates containing biphasic blood agar (Novy-Nicolle-McNeal) medium. The number of viable parasites was determined from the highest dilution at which promastigotes could be grown after up to 2 weeks of incubation at 25° C.

Following intradermal Leishmania amazonensis infection in the ear and lesion development, short-term intralesional treatment with H22-ETA halted disease progression in huCD64-transgenic mice but not in WT control littermates, used as controls for possible non-specific effects of the immunotoxin. As shown in FIG. 2a, b, c, H22-ETA treatment caused a four-fold decrease in lesion size (p=0.0017), a three-fold decrease in inflammation score (p=0.0052), as well as a five-fold decrease in parasite load (p=0.030). Notably, only 10 intradermal injections were sufficient to achieve a significant therapeutic effect in infected mice, which ascertains the in vivo applicability of the anti-CD64 IT, since patients typically receive 1 to 3 cycles of pentavalent antimonials, i.e. 20 to 60 intravenous injections to achieve clinical cure.

Legend to FIG. 2

CD64-directed immunotoxin demonstrates therapeutic potential in vivo. WT and HuCD64-Tg C57BL6 mice were infected intradermally in the right ear with 10⁵ Leishmania amazonensis stationary phase promastigotes. Following lesion development at 6 weeks post infection, both groups received 10 doses of 100 ng H22-ETA, intralesionally on alternate days. a, Lesion size was measured as right ear thickness, corrected for left ear values of each individual mouse (*p<0.05, **p<0.01, unpaired t test). b, Right ear inflammation was scored on a scale from 0 (absent) to 3 (severe), (**p=0.0052, Mann-Whitney test). c, Parasite load was determined by quantifying Leishmania promastigotes in serial dilutions of ear homogenates in Schneider's Insect Medium (*p=0.030, unpaired t test following log transformation).

Staining of Blood Cells, Recruitment and Follow Up of Patients

Peripheral blood samples (10 ml) were collected from patients and healthy controls (Salvador-Bahia urban area, no history of residence in endemic areas) by venipuncture using heparin as an anticoagulant. For whole-blood staining (cohort I), 50 μl of whole blood was diluted with an equal volume of PBS containing 1% BSA and 0.1% sodium azide, followed by staining for 30 min on ice with fluorescein-conjugated anti-CD64 (clone 22, Immunotech-Coulter, Marseille, France; 10.1, Pharmingen, BD Biosciences, US) and lineage markers CD14 (monocytes), CD3 (T cells), CD19 (B cells), CD16b (neutrophils), CD49d (eosinophils) and CD56 (NK cells) or isotype-matched control antibodies (all from Immunotech-Coulter, Marseille, France). Staining was followed by fixation and erythrocyte lysis (whole blood lysing solution, Becton-Dickinson, San Jose, Calif.). For PBMC staining (cohort II), 200,000 mononuclear cells (purified by Ficoll/Hypaque gradient centrifugation) were stained with the same antibody cocktails as cohort I. Since the drastic lysis and fixation step for whole blood was omitted, MFIs in cohort II were higher in both controls and patients. For each sample, 10,000-20,000 events were acquired in a flow cytometer (FACSort, Becton-Dickinson) and analyzed using CellQuest software. Monocytes were gated according to their characteristic forward-scatter and side-scatter as previously described⁹ and were confirmed to be CD14⁺, CD3⁻, CD19⁻, CD16b⁻ and CD56⁻.

Patients with cutaneous leishmaniasis from two consecutive cohorts were recruited and treated in two outpatient clinics (Jequié and Jiquiriçá, Bahia State, North-East Brazil) covering the same rural area, which has a low socio-economic status and a high incidence of infection with Leishmania braziliensis. This study was approved by the Ethics Committee of the University Hospital Professor Edgard Santos (first cohort, recruitment 2000-2001, follow-up until 2005) and of the Gonçalo Moniz Research Center (second cohort, recruitment 2002-2004, follow-up until 2006). Healthy controls (n=40) were analyzed in parallel within the same time frame and with the same staining protocol. Informed consent was obtained from all patients and healthy controls. A total of 53 patients provided blood samples of sufficient quality for flow cytometry analysis at the time of diagnosis with cutaneous leishmaniasis (as described⁹, according to characteristic lesion morphology, positive skin test, seropositivity towards Leishmania antigen and/or the presence of parasites in the lesion). Clinical and demographic data from both cohorts are listed in Table 2.

TABLE 2
Clinical characteristics of cutaneous leishmaniasis patients

| | | | Disease | Lesion | Healing | |
|---|---|---|---|---|---|---|
| | | Age | duration | diameter | time | Treatment |
| | Gender | (years) | (days) | (cm) | (days) | cycles |
| Cohort | 16M/5F | 31.3 ± 4.3 | 60 ± 7 | 2.5 ± 0.3 | 99 ± 22 | 1.04 ± 0.04 |
| I | | | | | | |
| Cohort | 16M/16F | 29.9 ± 2.8 | 37 ± 3 | 2.0 ± 0.2 | 142 ± 18 | 1.9 ± 0.2 |
| II | | | | | | |

To take both healing time (complete cicatrisation of lesions) and drug dosage (one cycle of standard treatment equals 20 days of intravenous pentavalent antimony) into account, therapeutic response was scored on a scale of 1-3 where 1 is fast (1 cycle and <60 d), 2 is intermediate (1-3 cycles and <360 d) and 3 is slow or non-healing (>3 cycles or >360 d). One patient form cohort I and five patients from cohort II did not have a complete two-year follow-up and were excluded from therapeutic response analysis.

Ex vivo expression of CD64 (FcγRI) in monocytes was significantly elevated in two independent CL cohorts. In the first cohort, CD64 mean fluorescence intensity (MFI) increased 2.4-fold in patients compared to healthy donors (p=0.0039; FIG. 3a), In the second cohort, a strikingly similar 2.7-fold increase in MFI was observed in patients vs. controls (p<0.0001; FIG. 3b). In addition, the percentage of CD64-positive cells was significantly increased in both cohorts (Supplementary FIG. 3a-b). CD64 expression was also analyzed in an additional group of 17 patients (second cohort) following standard antimonial therapy. As shown in FIG. 3c, the MFI of monocyte CD64 was reduced significantly following treatment, compared to patients before treatment (p=0.014). CD64 expression did not differ significantly between treated patients and controls (p=0.13), indicating that increased CD64 expression at diagnosis reflects disease status and is not an intrinsic feature of leishmaniasis patients. Moreover, monocyte surface levels of CD32 (FcγRII) were not significantly different before or after treatment (p=0.84, results not shown), indicating a selective up-regulation of CD64 during active disease in human CL. CD64 MFI positively correlated to therapeutic failure in the first cohort (Supplementary FIG. 3c, r=0.65, p=0.0018). This selective association between CD64 and therapeutic response was validated in a second larger cohort (Supplementary FIG. 3d, r=0.51, p=0.006). Since the fast, intermediate and slow/non-healing phenotypes were similarly distributed in both cohorts, a joint analysis was possible following normalization of CD64 MFI. As shown in FIG. 3d, normalized CD64 expression was strongly associated with therapeutic response (p<0.0001). Both fast vs. slow healing patients (area under the ROC curve 0.97, p=0.0004, sensitivity 95.8% (95% CI [78.9-99.9%]), specificity 83.3% (95% CI [35.9-99.6%], likelihood ratio 5.8) and fast vs. intermediate and slow healing patients (area under the ROC curve 0.81, p=0.0003, sensitivity 66.7% (95% CI [44.7-84.4%], specificity 90.5% (95% CI [69.6-98.8%], likelihood ratio 7.0) could be significantly discriminated through their CD64 level at diagnosis, thus authenticating its clinical value as a biomarker.

Legend to FIG. 3

CD64 is a biomarker for therapeutic response in cutaneous leishmaniasis. a, b, Flow cytometric analysis of ex vivo monocyte CD64 expression (as mean fluorescence intensity, MFI), in 53 cutaneous leishmaniasis (CL) patients (cohort I and II, **p=0.0039, ***p<0.0001, respectively, t test with Welch's correction) and 40 normal donors. c, Flow cytometric analysis of ex vivo monocyte CD64 expression (MFI) in CL patients before and after treatment (cohort II, *p=0.014, t test with Welch's correction). d, Normalized CD64 levels for both cohorts of CL patients classified according to therapeutic response (fast, intermediate or slow healing as described in Supplementary Methods, ANOVA p<0.0001, post-test for linear trend p<0.0001). This study was approved by the Ethics Committee of the University Hospital Professor Edgard Santos (first cohort, recruitment 2000-2001, follow-up until 2005) and of the Gonçalo Moniz Research Center (second cohort, recruitment 2002-2004, follow-up until 2006).

List of Amino Acid Sequences (Single Letter Code of Amino Acids)

Sequence 1: H22(scFv)-ETA′
MKYLLPTAAAGLLLLAAQPAMAMGHHHHHHHHHHSSGHIDDDDKHMKLMAQPAMAQVQLVESGGGVVQ
PGRSLRLSCSSSGFIFSDNYMYWVRQAPGKGLEWVATISDGGSYTYYPDSVKGRFTISRDNSKNTLFL
QMDSLRPEDTGVYFCARGYYRYEGAMDYWGQGTPVTVSSGGGGSGGGGSGGGGSDIQLTQSPSSLSAS
VGDRVTITCKSSQSVLYSSNQKNYLAWYQQKPGKAPKLLIYWASTRESGVPSRFSGSGSGTDFTFTIS
SLQPEDIATYYCHQYLSSWTFGQGTKLEIKAAAELASGGPEGGSLAALTAHQACHLPLETFTRHRQPR
GWEQLEQCGYPVQRLVALYLAARLSWNQVDQVIRNALASPGSGGDLGEAIREQPEQARLALTLAAAES
ERFVRQGTGNDEAGAANADVVSLTCPVAAGECAGPADSGDALLERNYPTGAEFLGDGGDVSFSTRGTQ
NWTVERLLQAHRQLEERGYVFVGYHGTFLEAAQSIVFGGVRARSQDLDAIWRGFYIAGDPALAYGYAQ
DQEPDARGRIRNGALLRVYVPRSSLPGFYRTGLTLAAPEAAGEVERLIGHPLPLRLDAITGPEEEGGR
LETILGWPLAERTVVIPSAIPTDPRNVGGDLDPSSIPDKEQAISALPDYASQPGKPPREDLK
Sequence 2: H22(scFv)-Ang
METDTLLLWVLLLWVPGSTGDAAQPAMAQVQLVESGGGVVQPGRSLRLSCSSSGFIFSDNYMYWVRQA
PGKGLEWVATISDGGSYTYYPDSVKGRFTISRDNSKNTLFLQMDSLRPEDTGVYFCARGYYRYEGAMD
YWGQGTPVTVSSGGGGSGGGGSGGGGSDIQLTQSPSSLSASVGDRVTITCKSSQSVLYSSNQKNYLAW
YQQKPGKAPKLLIYWASTRESGVPSRFSGSGSGTDFTFTISSLQPEDIATYYCHQYLSSWTFGQGTKL
EIKAAALESRQDNSRYTHFLTQHYDAKPQGRDDRYCESIMRRRGLTSPCKDINTFIHGNKRSIKAICE
NKNGNPHRENLRISKSSFQVTTCKLHGGSPWPPCQYRATAGFRNVVVACENGLPVHLDQSIFRRPAEH
EFRGGPEQKLISEEDLNSAVDHHHHHH
Sequence 3: H22(scFv)-_{cat}AD-Ang
METDTLLLWVLLLWVPGSTGDAAQPAMAQVQLVESGGGVVQPGRSLRLSCSSSGFIFSDNYMYWVRQA
PGKGLEWVATISDGGSYTYYPDSVKGRFTISRDNSKNTLFLQMDSLRPEDTGVYFCARGYYRYEGAMD
YWGQGTPVTVSSGGGGSGGGGSGGGGSDIQLTQSPSSLSASVGDRVTITCKSSQSVLYSSNQKNYLAW
YQQKPGKAPKLLIYWASTRESGVPSRFSGSGSGTDFTFTISSLQPEDIATYYCHQYLSSWTFGQGTKL
EIKAAAGGGGSALALPLSSIFSRIGDPGGPYVHDEVDRGPPGSRQDNSRYTHFLTQHYDAKPQGRDDR
YCESIMRRRGLTSPCKDINTFIHGNKRSIKAICENKNGNPHRENLRISKSSFQVTTCKLHGGSPWPPC
QYRATAGFRNVVVACENGLPVHLDQSIFRRPAEHEFRGGPEQKLISEEDLNSAVDHHHHHH
Sequence 4: H22(scFv)-Ann_{GGRR}
METDTLLLWVLLLWVPGSTGDAAQPAMAQVQLVESGGGVVQPGRSLRLSCSSSGFIFSDNYMYWVRQA
PGKGLEWVATISDGGSYTYYPDSVKGRFTISRDNSKNTLFLQMDSLRPEDTGVYFCARGYYRYEGAMD
YWGQGTPVTVSSGGGGSGGGGSGGGGSDIQLTQSPSSLSASVGDRVTITCKSSQSVLYSSNQKNYLAW
YQQKPGKAPKLLIYWASTRESGVPSRFSGSGSGTDFTFTISSLQPEDIATYYCHQYLSSWTFGQGTKL
EIKAAALESRQDNSRYTHFLTQHYDAKPGGRRDRYCESIMRRRGLTSPCKDINTFIHGNKRSIKAICE
NKNGNPHRENLRISKSSFQVTTCKLHGGSPWPPCQYRATAGFRNVVVACENGLPVHLDQSIFRRPAEH
EFRGGPEQKLISEEDLNSAVDHHHHHH
Sequence 5: H22(scFv)-_{cat}AD-Ann_{GGRR}
METDTLLLWVLLLWVPGSTGDAAQPAMAQVQLVESGGGVVQPGRSLRLSCSSSGFIFSDNYMYWVRQA
PGKGLEWVATISDGGSYTYYPDSVKGRFTISRDNSKNTLFLQMDSLRPEDTGVYFCARGYYRYEGAMD
YWGQGTPVTVSSGGGGSGGGGSGGGGSDIQLTQSPSSLSASVGDRVTITCKSSQSVLYSSNQKNYLAW
YQQKPGKAPKLLIYWASTRESGVPSRFSGSGSGTDFTFTISSLQPEDIATYYCHQYLSSWTFGQGTKL
EIKAAAGGGGSALALPLSSIFSRIGDPGGPYVHDEVDRGPPGSRQDNSRYTHFLTQHYDAKPGGRRDR
YCESIMRRRGLTSPCKDINTFIHGNKRSIKAICENKNGNPHRENLRISKSSFQVTTCKLHGGSPWPPC
QYRATAGFRNVVVACENGLPVHLDQSIFRRPAEHEFRGGPEQKLISEEDLNSAVDHHHHHH
Sequence 6: H22(scFv)-_{cat}AD-GB
METDTLLLWVLLLWVPGSTGDAAQPAMAQVQLVESGGGVVQPGRSLRLSCSSSGFIFSDNYMYWVRQA
PGKGLEWVATISDGGSYTYYPDSVKGRFTISRDNSKNTLFLQMDSLRPEDTGVYFCARGYYRYEGAMD
YWGQGTPVTVSSGGGGSGGGGSGGGGSDIQLTQSPSSLSASVGDRVTITCKSSQSVLYSSNQKNYLAW
YQQKPGKAPKLLIYWASTRESGVPSRFSGSGSGTDFTFTISSLQPEDIATYYCHQYLSSWTFGQGTKL
EIKAAAGGGGSALALPLSSIFSRIGDPGGPYVHDEVDRGPIIGGHEAKPHSRPYMAFLMIWDQKSLKR
CGGFLIRDDFVLTAAHCWGSSINVTLGAHNIKEQEPTQQFIPVKRAIPHPAYNPKNFSNDIMLLQLER
KAKRTRAVQPLRLPSNKAQVKPGQTCSVAGWGQTAPLGKHSHTLQEVKMTVQEDRKCESDLRHYYDST
IELCVGDPEIKKTSFKGDSGGPLVCNKVAQGIVSYGRNNGMPPRACTKVSSFVHWIKKTMKRYAEHHH
HHH
Sequence 7: H22(scFv)-_{cat}AD-GB_{R201K}
METDTLLLWVLLLWVPGSTGDAAQPAMAQVQLVESGGGVVQPGRSLRLSCSSSGFIFSDNYMYWVRQA
PGKGLEWVATISDGGSYTYYPDSVKGRFTISRDNSKNTLFLQMDSLRPEDTGVYFCARGYYRYEGAMD
YWGQGTPVTVSSGGGGSGGGGSGGGGSDIQLTQSPSSLSASVGDRVTITCKSSQSVLYSSNQKNYLAW
YQQKPGKAPKLLIYWASTRESGVPSRFSGSGSGTDFTFTISSLQPEDIATYYCHQYLSSWTFGQGTKL
EIKAAAGGGGSALALPLSSIFSRIGDPGGPYVHDEVDRGPIIGGHEAKPHSRPYMAFLMIWDQKSLKR
CGGFLIRDDFVLTAAHCWGSSINVTLGAHNIKEQEPTQQFIPVKRAIPHPAYNPKNFSNDIMLLQLER
KAKRTRAVQPLRLPSNKAQVKPGQTCSVAGWGQTAPLGKHSHTLQEVKMTVQEDRKCESDLRHYYDST
IELCVGDPEIKKTSFKGDSGGPLVCNKVAQGIVSYGKNNGMPPRACTKVSSFVHWIKKTMKRYAEHHH
HHH
Sequence 8: H22(scFv)-MAP
HHHHHHHHHHSSGHIDDDDKHMKLMAQPAMAQVQLVESGGGVVQPGRSLRLSCSSSGFIFSDNYMYWV
RQAPGKGLEWVATISDGGSYTYYPDSVKGRFTISRDNSKNTLFLQMDSLRPEDTGVYFCARGYYRYEG
AMDYWGQGTPVTVSSGGGGSGGGGSGGGGSDIQLTQSPSSLSASVGDRVTITCKSSQSVLYSSNQKNY
LAWYQQKPGKAPKLLIYWASTRESGVPSRFSGSGSGTDFTFTISSLQPEDIATYYCHQYLSSWTFGQG
TKLEIKAAAMAEPRQEFEVMEDHAGTYGLGDRKDQGGYTMHQDQEGDTDAGLKAEEAGIGDTPSLEDE
AAGHVTQARMVSKSKDGTGSDDKKAKGADGKTKIATPRGAAPPGQKGQANATRIPAKTPPAPKTPPSS
GEPPKSGDRSGYSSPGSPGTPGSRSRTPALPTPPTREPKKVAVVRTPPKSPSSAKSRLQTAPVPMPDL
KNVKSKIGATENLKHQPGGGKVQIINKKLDLSNVQSKCGSKDNIKHVPGGGSVQIVYKPVDLSKVTSK
CGSLGNIHHKPGGGQVEVKSEKLDFKDRVQSKIGSLDNITHVPGGGNKKIETHKLTFRENAKAKTDHG
AEIVYKSPVVSGDTSPRHLSNVSSTGSIDMVDSPQLATLADEVSASLAKQGLPKKKRKV
Sequence 9: GB-H22(scFv)
HIDDDDKIIGGHEAKPHSRPYMAFLMIWDQKSLKRCGGFLIRDDFVLTAAHCWGSSINVTLGAHNIKE
QEPTQQFIPVKRAIPHPAYNPKNFSNDIMLLQLERKAKRTRAVQPLRLPSNKAQVKPGQTCSVAGWGQ
TAPLGKHSHTLQEVKMTVQEDRKCESDLRHYYDSTIELCVGDPEIKKTSFKGDSGGPLVCNKVAQGIV
SYGRNNGMPPRACTKVSSFVHWIKKTMKRYGSKLAEHEGDAAQPAMAQVQLVESGGGVVQPGRSLRLS
CSSSGFIFSDNYMYWVRQAPGKGLEWVATISDGGSYTYYPDSVKGRFTISRDNSKNTLFLQMDSLRPE
DTGVYFCARGYYRYEGAMDYWGQGTPVTVSSGGGGSGGGGSGGGGSDIQLTQSPSSLSASVGDRVTIT
CKSSQSVLYSSNQKNYLAWYQQKPGKAPKLLIYWASTRESGVPSRFSGSGSGTDFTFTISSLQPEDIA
TYYCHQYLSSWTFGQGTKLEIKAAAGPHHHHHH
Sequence 10: GB_{R201K}-H22(scFv)
HIDDDDKIIGGHEAKPHSRPYMAFLMIWDQKSLKRCGGFLIRDDFVLTAAHCWGSSINVTLGAHNIKE
QEPTQQFIPVKRAIPHPAYNPKNFSNDIMLLQLERKAKRTRAVQPLRLPSNKAQVKPGQTCSVAGWGQ
TAPLGKHSHTLQEVKMTVQEDRKCESDLRHYYDSTIELCVGDPEIKKTSFKGDSGGPLVCNKVAQGIV
SYGKNNGMPPRACTKVSSFVHWIKKTMKRYAEHEGDAAQPAMAQVQLVESGGGVVQPGRSLRLSCSSS
GFIFSDNYMYWVRQAPGKGLEWVATISDGGSYTYYPDSVKGRFTISRDNSKNTLFLQMDSLRPEDTGV
YFCARGYYRYEGAMDYWGQGTPVTVSSGGGGSGGGGSGGGGSDIQLTQSPSSLSASVGDRVTITCKSS
QSVLYSSNQKNYLAWYQQKPGKAPKLLIYWASTRESGVPSRFSGSGSGTDFTFTISSLQPEDIATYYC
HQYLSSWTFGQGTKLEIKAAAGPHHHHHH
Sequence 11: GM-H22(scFv)
HIDDDDKIIGGREVIPHSRPYMASLQRNGSHLCGGVLVHPKWVLTAAHCLAQRMAQLRLVLGLHTLDS
PGLTFHIKAAIQHPRYKPVPALENDLALLQLDGKVKPSRTIRPLALPSKRQVVAAGTRCSMAGWGLTH
QGGRLSRVLRELDLQVLDTRMCNNSRFWNGSLSPSMVCLAADSKDQAPCKGDSGGPLVCGKGRVLAGV
LSFSSRVCTDIFKPPVATAVAPYVSWIRKVTGRSAAEHEGDAAQPAMAQVQLVESGGGVVQPGRSLRL
SCSSSGFIFSDNYMYWVRQAPGKGLEWVATISDGGSYTYYPDSVKGRFTISRDNSKNTLFLQMDSLRP
EDTGVYFCARGYYRYEGAMDYWGQGTPVTVSSGGGGSGGGGSGGGGSDIQLTQSPSSLSASVGDRVTI
TCKSSQSVLYSSNQKNYLAWYQQKPGKAPKLLIYWASTRESGVPSRFSGSGSGTDFTFTISSLQPEDI
ATYYCHQYLSSWTFGQGTKLEIKAAAGPHHHHHH

### REFERENCES

[1] P. Desjeux, “Leishmaniasis: current situation and new perspectives.,” Comp. Immunol. Microbiol. Infect. Dis., vol. 27, no. 5, pp. 305-318, September 2004.
[2] P. Desjeux, “Leishmaniasis.,” Nature reviews. Microbiology, vol. 2, no. 9, p. 692, September-2004.
[3] H. W. Murray, J. D. Berman, C. R. Davies, and N. G. Saravia, “Advances in leishmaniasis.,” Lancet, vol. 366, no. 9496, pp. 1561-1577, November 2005.
[4] F. Modabber, P. A. Buffet, E. Torreele, G. Milon, and S. L. Croft, “Consultative meeting to develop a strategy for treatment of cutaneous leishmaniasis. Institute Pasteur, Paris. 13-15 June, 2006.,” Kinetoplastid Biol Dis, vol. 6, p. 3, 2007.
[5] J. C. Leemans, T. Thepen, S. Weijer, S. Florquin, N. van Rooijen, J. G. van de Winkel, and T. van der Poll, “Macrophages play a dual role during pulmonary tuberculosis in mice.,”J Infect Dis, vol. 191, no. 1, pp. 65-74, January 2005.
[6] E. Peterson, S. M. Owens, and R. L. Henry, “Monoclonal antibody form and function: manufacturing the right antibodies for treating drug abuse.,” AAPS J, vol. 8, no. 2, pp. E383-90, 2006.
[7] B. Matthey, A. Engert, A. Klimka, V. Diehl, and S. Barth, “A new series of pET-derived vectors for high efficiency expression of Pseudomonas exotoxin-based fusion proteins.,” Gene, vol. 229, no. 1, pp. 145-153, March 1999.
[8] M. R. Green and J. Sambrook, Molecular Cloning. 2012.
[9] B. Stahnke, T. Thepen, M. Stöcker, R. Rosinke, E. Jost, R. Fischer, M. K. Tur, and S. Barth, “Granzyme B-H22(scFv), a human immunotoxin targeting CD64 in acute myeloid leukemia of monocytic subtypes,” Mol. Cancer Ther., vol. 7, no. 9, pp. 2924-2932, September 2008.
[10] M. K. Tur, M. Huhn, T. Thepen, M. Stöcker, R. Krohn, S. Vogel, E. Jost, R. Osieka, J. G. van de Winkel, R. Fischer, R. Finnern, and S. Barth, “Recombinant CD64-specific single chain immunotoxin exhibits specific cytotoxicity against acute myeloid leukemia cells.,” Cancer Res, vol. 63, no. 23, pp. 8414-8419, December 2003.
[11] T. Thepen, A. J. van Vuuren, R. C. Kiekens, C. A. Damen, W. C. Vooijs, and J. G. Van De Winkel, “Resolution of cutaneous inflammation after local elimination of macrophages.,” Nat. Biotechnol., vol. 18, no. 1, pp. 48-51, January 2000.
[12] I. A. I. Heijnen, I. A. I. Heijnen, J. G. J. Van de Winkel, and J. G. J. Van de Winkel, “A human Fc gamma RI/CD64 transgenic model for in vivo analysis of (bispecific) antibody therapeutics.,”J Hematother, vol. 4, no. 5, pp. 351-356, September 1995.

1. An immunotoxin for use in the treatment of leishmaniasis wherein the immunotoxin comprises a portion which is specifically binding to the cellular surface receptor CD64 as a component A and a cell killing portion as a component B, wherein the cell killing portion alters the function, gene expression, or viability of a cell thereby killing Leishmania-infected macrophages and by this eliminates Leishmania.

2. The immunotoxin of claim 1 wherein the cell killing portion is covalently bonded to the portion specifically binding to the cellular surface receptor CD64.

3. The immunotoxin of claim 1 wherein the immunotoxin is a recombinant protein or the portion specifically binding to the cellular surface receptor CD64 is linked directly to the cell killing portion or linked via a linking group.

4. The immunotoxin of claim 1 wherein the portion which is specifically binding to the cellular surface receptor CD64 is selected from the group consisting of antibodies or their derivatives or fragments, such as scFv fragments; synthetic peptides or molecules; ligands; receptor binding molecules, and their structural analogs; mutants and combinations thereof.

5. The immunotoxin of claim 1 wherein the portion which is specifically binding to the cellular surface receptor CD64 is a recombinant molecule.

6. The immunotoxin of claim 1 wherein the cell killing portion alters the function, gene expression, or viability of a cell by inactivating molecules responsible for protein biosynthesis or activating components of cell-inherent apoptosis pathways.

7. The immunotoxin of claim 1 wherein the cell killing portion is cytotoxic in particular a molecule selected from the group consisting of a member of ADP-ribosylating enzymes, such as the Pseudomonas Exotoxin A, Diphtheria-, Cholera- or the Pertussis-, Botulinumtoxin; a ribosome-inactivating protein such as Dianthin, Saporin, Bryodin, Gelonin, Ricin, Abrin, Pokeweed Antiviral Protein (PAP) or Restrictocin; or is a member of the RNases (Phosphodiesterases) such as the Bovine seminal RNase, BovineRNase A, Bovine pancreatic RNase, Angiogenin, Eosinophil-derived Neurotoxin (EDN), Eosinophilic Cationic Protein (ECP), Onconase, or Bullfrog Lectin; a prodrug-activating enzyme such as Calicheamicin, Glucose Oxidase, Carboxypeptidase, Alkaline Phosphatase, Cytosindeaminase, β-Glucosidase, β-Glucuronidase, β-Lactamase, Nitroreductase, Thymidinkinase or Purin Nukleosid Phosphorylase; a cathepsin protease; a calpain; or a granzyme; a microtubule-binding protein including tau; any derivative of the above mentioned proteins; and combinations thereof.

8. The immunotoxin of claim 7 wherein the cell killing portion is a molecule selected from the group consisting an of ADP-ribosylating enzyme and ribosome-inactivating protein.

9. The immunotoxin of claim 1 wherein the cell killing portion is a small molecule selected from the group of alkylating agents (e.g. cyclophosphamide, cholrambucil), anthracyclins (doxorubicin, daunomycin), maytansinoids (maytansinoid DM1), anti-metabolites, plant alkaloids and terpenoids as the Vinca alkaloids (vinblastine, vincristine vinorebline, vindesin) Podophyllotoxin and structural analogs hereof and taxanes (paclitaxel, docetaxel, taxotere) or topoisomerase inhibitors (camptothecins), synthetic toxins as ellipticine analogs or synthetic analogs of tumor antibiotics as duocarmycin or CC1065, other tubulin binding agents as halichondrin B, hemiasterlins and dolastatins or analogs as monomethyl-auristatin E; or a component B is selected from the group of small molecules having cytotoxic/cytostatic activities like alkylating agents (like Cyclophosphamide, Mechlorethamine, Chlorambucil, Melphalan), anthracyclines (like Danorubicin, Doxorubicin, Epirubicin, Idarubicin, Mitoxantrone, Valrubicin), cytoskeletal disruptors (like Paclitaxel, Docetaxel) or Epothilones, Inhibitors of topoisomerase II (like Etoposide, Teniposide, Tafluposide), nucleotide analogs and precursor analogs (like azacididine, azathioprine, capecitabine, cytarabine, doxofluridine, fluorouracil, gemcitabine, mercaptopurine, methotrexate, tioguanine), peptide antibiotics (like bleomycin), platinum-based agents (like carboplatin, cisplatin, oxaliplatin), retinoids (like all-trans retinoic acid), vinca alkaloids and structural analogs (like vinblastine, vincristine, vindestine, vinorelbine), beta ray emitting nuclides like Iodine-131, Yttrium-90, Lutetium-177, Aromatase Inhibitors (like Aminoglutethimide, Anastrozole, Letrozole, Vorozole, Exemestane, 4-androstene-3,6,17-trione, 1,4,6-androstatrien-3,17-dione, Formestane, Testolactone), Carbonic Anhydrase Inhibitors (like Acetazolamide, Methazolamide, Dorzolamide, Topiramate), Cholinesterase Inhibitors (Organophosphates like Metrifonate, Carbamates like Physostigmine, Neostigmine, Pyridostigmine, Ambenonium, Demarcarium, Rivastigmine, Phananthrine like Galantamine, Piperidine like Donepezil, Tacrine, Edophonium, or Phenothiazines), Cyclooxygenase Inhibitors (like Celecoxib, Rofecoxib, Etoricoxib, Acetaminophen, Diclofenac, Ibuprofen), Folic Acid Antagonists (like Methotrexate), Hydroxymethylglutaryl-CoA Reductase Inhibitors (like Atorvastatin, Cerivastatin, Fluvastatin, Lovastatin, Mevastatin, Pitavastatin, Pravastatin, Rosuvastatin, Simvastatin, Vytorin, Advicor, Caduet), Integrase Inhibitors (like Raltegravir, Elvitegravir), Lipoxygenase Inhibitors (like Zileutron), Monoamine Oxidase Inhibitors (like Isocarboxazid, Moclobemide, Phenelzine, Tranylcypromine, Selegiline, Rasagiline, Nialamide, Iproniazid, Iproclozide, Toloxatone, Linezolid, Tryptamines, Dienolide, Detxtroamphetamine), Nucleic Acid Synthesis Inhibitors, Phosphodiesterase Inhibitors (like Caffeine, Theopyline, 3-isobutyl-1-methylxanthine, Vinpocetine, EHNA, Enoximone, Lirinone, PDE3, Mesembrine, Rolipram, Ibudilast, Sildenafil, Tadalafil, Vardenafil, Udenafil, Avanafil), Protease Inhibitors (like Saquinavir, Ritonavir, Idinavir, Nelfinavir, Amprenavir, Lopinavir, Atazanavir, Fosamprenavir, Tipranavir, Darunavir), Protein Kinase Inhibitors (like Imatinib, Geftinib, Pegaptanib, Sorafenib, Dasatinib, Sunitinib, Erlotinib, Nilotinib, Lapatinib), Protein Synthesis Inhibitors (like Anisomycin, Cycloheximide, Chloramphenicol, Tetracycline, Streptomycin, Erythromycin, Puromycin, etc.), Proton Pump Inhibitors (like Omeprazole, Lansoprazole, Esomeprazole, Pantoprazole, Rabeprazole), oligonucleotides, nucleic acids like small interfering RNAs (siRNAs), a short hairpin RNA (shRNA), an antisense DNA or RNA, a double stranded RNA (dsRNA) and a micro RNA (miRNA) might be used to down-regulate specific key elements of regulative pathways within a cell.

10. The immunotoxin of claim 9 wherein the cell killing portion is a molecule selected from the group of Pseudomonas Exotoxin A and Ricin.

11. The immunotoxin of claim 1 having at least one supplementary component C.

12. The immunotoxin of claim 11 wherein the component C regulates expression of a gene encoding the immunotoxin.

13. The immunotoxin according to claim 11 wherein component C enables purification of the recombinant immunotoxin or its individual component A or B.

14. The immunotoxin according to claim 11 wherein the component C stimulates internalization of the immunotoxin or its individual components, in particular of the cell killing portion, into a macrophage as target cell.

15. The immunotoxin according to claim 11 wherein the component C triggers translocation of the cell killing portion into a subcellular compartment.

16. The immunotoxin according to claim 11 wherein the component C stimulates proteolytic removal of the portion which is specifically binding to the cellular surface receptor CD64 from the cell killing portion.

17. The immunotoxin according to claim 11 wherein the component C triggers intracellular activation of the cell killing portion.


SEQUENCE LISTING (11 sequences)

SEQ ID NO: 1 - protein, 674 aa, artificial
  fusion protein
MKYLLPTAAAGLLLLAAQPAMAMGHHHHHHHHHHSSGHIDDDDKHMKLMAQPAMAQVQLVESGGGVVQPGRSLRLSCSSSGFIFSDNYMYWVRQAPGKGLEWVATISDGGSYTYYPDSVKGRFTISRDNSKNTLFLQMDSLRPEDTGVYFCARGYYRYEGAMDYWGQGTPVTVSSGGGGSGGGGSGGGGSDIQLTQSPSSLSASVGDRVTITCKSSQSVLYSSNQKNYLAWYQQKPGKAPKLLIYWASTRESGVPSRFSGSGSGTDFTFTISSLQPEDIATYYCHQYLSSWTFGQGTKLEIKAAAELASGGPEGGSLAALTAHQACHLPLETFTRHRQPRGWEQLEQCGYPVQRLVALYLAARLSWNQVDQVIRNALASPGSGGDLGEAIREQPEQARLALTLAAAESERFVRQGTGNDEAGAANADVVSLTCPVAAGECAGPADSGDALLERNYPTGAEFLGDGGDVSFSTRGTQNWTVERLLQAHRQLEERGYVFVGYHGTFLEAAQSIVFGGVRARSQDLDAIWRGFYIAGDPALAYGYAQDQEPDARGRIRNGALLRVYVPRSSLPGFYRTGLTLAAPEAAGEVERLIGHPLPLRLDAITGPEEEGGRLETILGWPLAERTVVIPSAIPTDPRNVGGDLDPSSIPDKEQAISALPDYASQPGKPPREDLK

SEQ ID NO: 2 - protein, 435 aa, artificial
  fusion protein
METDTLLLWVLLLWVPGSTGDAAQPAMAQVQLVESGGGVVQPGRSLRLSCSSSGFIFSDNYMYWVRQAPGKGLEWVATISDGGSYTYYPDSVKGRFTISRDNSKNTLFLQMDSLRPEDTGVYFCARGYYRYEGAMDYWGQGTPVTVSSGGGGSGGGGSGGGGSDIQLTQSPSSLSASVGDRVTITCKSSQSVLYSSNQKNYLAWYQQKPGKAPKLLIYWASTRESGVPSRFSGSGSGTDFTFTISSLQPEDIATYYCHQYLSSWTFGQGTKLEIKAAALESRQDNSRYTHFLTQHYDAKPQGRDDRYCESIMRRRGLTSPCKDINTFIHGNKRSIKAICENKNGNPHRENLRISKSSFQVTTCKLHGGSPWPPCQYRATAGFRNVVVACENGLPVHLDQSIFRRPAEHEFRGGPEQKLISEEDLNSAVDHHHHHH

SEQ ID NO: 3 - protein, 469 aa, artificial
  fusion protein
METDTLLLWVLLLWVPGSTGDAAQPAMAQVQLVESGGGVVQPGRSLRLSCSSSGFIFSDNYMYWVRQAPGKGLEWVATISDGGSYTYYPDSVKGRFTISRDNSKNTLFLQMDSLRPEDTGVYFCARGYYRYEGAMDYWGQGTPVTVSSGGGGSGGGGSGGGGSDIQLTQSPSSLSASVGDRVTITCKSSQSVLYSSNQKNYLAWYQQKPGKAPKLLIYWASTRESGVPSRFSGSGSGTDFTFTISSLQPEDIATYYCHQYLSSWTFGQGTKLEIKAAAGGGGSALALPLSSIFSRIGDPGGPYVHDEVDRGPPGSRQDNSRYTHFLTQHYDAKPQGRDDRYCESIMRRRGLTSPCKDINTFIHGNKRSIKAICENKNGNPHRENLRISKSSFQVTTCKLHGGSPWPPCQYRATAGFRNVVVACENGLPVHLDQSIFRRPAEHEFRGGPEQKLISEEDLNSAVDHHHHHH

SEQ ID NO: 4 - protein, 435 aa, artificial
  fusion protein
METDTLLLWVLLLWVPGSTGDAAQPAMAQVQLVESGGGVVQPGRSLRLSCSSSGFIFSDNYMYWVRQAPGKGLEWVATISDGGSYTYYPDSVKGRFTISRDNSKNTLFLQMDSLRPEDTGVYFCARGYYRYEGAMDYWGQGTPVTVSSGGGGSGGGGSGGGGSDIQLTQSPSSLSASVGDRVTITCKSSQSVLYSSNQKNYLAWYQQKPGKAPKLLIYWASTRESGVPSRFSGSGSGTDFTFTISSLQPEDIATYYCHQYLSSWTFGQGTKLEIKAAALESRQDNSRYTHFLTQHYDAKPGGRRDRYCESIMRRRGLTSPCKDINTFIHGNKRSIKAICENKNGNPHRENLRISKSSFQVTTCKLHGGSPWPPCQYRATAGFRNVVVACENGLPVHLDQSIFRRPAEHEFRGGPEQKLISEEDLNSAVDHHHHHH

SEQ ID NO: 5 - protein, 469 aa, artificial
  fusion protein
METDTLLLWVLLLWVPGSTGDAAQPAMAQVQLVESGGGVVQPGRSLRLSCSSSGFIFSDNYMYWVRQAPGKGLEWVATISDGGSYTYYPDSVKGRFTISRDNSKNTLFLQMDSLRPEDTGVYFCARGYYRYEGAMDYWGQGTPVTVSSGGGGSGGGGSGGGGSDIQLTQSPSSLSASVGDRVTITCKSSQSVLYSSNQKNYLAWYQQKPGKAPKLLIYWASTRESGVPSRFSGSGSGTDFTFTISSLQPEDIATYYCHQYLSSWTFGQGTKLEIKAAAGGGGSALALPLSSIFSRIGDPGGPYVHDEVDRGPPGSRQDNSRYTHFLTQHYDAKPGGRRDRYCESIMRRRGLTSPCKDINTFIHGNKRSIKAICENKNGNPHRENLRISKSSFQVTTCKLHGGSPWPPCQYRATAGFRNVVVACENGLPVHLDQSIFRRPAEHEFRGGPEQKLISEEDLNSAVDHHHHHH

SEQ ID NO: 6 - protein, 547 aa, artificial
  fusion protein
METDTLLLWVLLLWVPGSTGDAAQPAMAQVQLVESGGGVVQPGRSLRLSCSSSGFIFSDNYMYWVRQAPGKGLEWVATISDGGSYTYYPDSVKGRFTISRDNSKNTLFLQMDSLRPEDTGVYFCARGYYRYEGAMDYWGQGTPVTVSSGGGGSGGGGSGGGGSDIQLTQSPSSLSASVGDRVTITCKSSQSVLYSSNQKNYLAWYQQKPGKAPKLLIYWASTRESGVPSRFSGSGSGTDFTFTISSLQPEDIATYYCHQYLSSWTFGQGTKLEIKAAAGGGGSALALPLSSIFSRIGDPGGPYVHDEVDRGPIIGGHEAKPHSRPYMAFLMIWDQKSLKRCGGFLIRDDFVLTAAHCWGSSINVTLGAHNIKEQEPTQQFIPVKRAIPHPAYNPKNFSNDIMLLQLERKAKRTRAVQPLRLPSNKAQVKPGQTCSVAGWGQTAPLGKHSHTLQEVKMTVQEDRKCESDLRHYYDSTIELCVGDPEIKKTSFKGDSGGPLVCNKVAQGIVSYGRNNGMPPRACTKVSSFVHWIKKTMKRYAEHHHHHH

SEQ ID NO: 7 - protein, 547 aa, artificial
  fusion protein
METDTLLLWVLLLWVPGSTGDAAQPAMAQVQLVESGGGVVQPGRSLRLSCSSSGFIFSDNYMYWVRQAPGKGLEWVATISDGGSYTYYPDSVKGRFTISRDNSKNTLFLQMDSLRPEDTGVYFCARGYYRYEGAMDYWGQGTPVTVSSGGGGSGGGGSGGGGSDIQLTQSPSSLSASVGDRVTITCKSSQSVLYSSNQKNYLAWYQQKPGKAPKLLIYWASTRESGVPSRFSGSGSGTDFTFTISSLQPEDIATYYCHQYLSSWTFGQGTKLEIKAAAGGGGSALALPLSSIFSRIGDPGGPYVHDEVDRGPIIGGHEAKPHSRPYMAFLMIWDQKSLKRCGGFLIRDDFVLTAAHCWGSSINVTLGAHNIKEQEPTQQFIPVKRAIPHPAYNPKNFSNDIMLLQLERKAKRTRAVQPLRLPSNKAQVKPGQTCSVAGWGQTAPLGKHSHTLQEVKMTVQEDRKCESDLRHYYDSTIELCVGDPEIKKTSFKGDSGGPLVCNKVAQGIVSYGKNNGMPPRACTKVSSFVHWIKKTMKRYAEHHHHHH

SEQ ID NO: 8 - protein, 671 aa, artificial
  fusion protein
HHHHHHHHHHSSGHIDDDDKHMKLMAQPAMAQVQLVESGGGVVQPGRSLRLSCSSSGFIFSDNYMYWVRQAPGKGLEWVATISDGGSYTYYPDSVKGRFTISRDNSKNTLFLQMDSLRPEDTGVYFCARGYYRYEGAMDYWGQGTPVTVSSGGGGSGGGGSGGGGSDIQLTQSPSSLSASVGDRVTITCKSSQSVLYSSNQKNYLAWYQQKPGKAPKLLIYWASTRESGVPSRFSGSGSGTDFTFTISSLQPEDIATYYCHQYLSSWTFGQGTKLEIKAAAMAEPRQEFEVMEDHAGTYGLGDRKDQGGYTMHQDQEGDTDAGLKAEEAGIGDTPSLEDEAAGHVTQARMVSKSKDGTGSDDKKAKGADGKTKIATPRGAAPPGQKGQANATRIPAKTPPAPKTPPSSGEPPKSGDRSGYSSPGSPGTPGSRSRTPALPTPPTREPKKVAVVRTPPKSPSSAKSRLQTAPVPMPDLKNVKSKIGATENLKHQPGGGKVQIINKKLDLSNVQSKCGSKDNIKHVPGGGSVQIVYKPVDLSKVTSKCGSLGNIHHKPGGGQVEVKSEKLDFKDRVQSKIGSLDNITHVPGGGNKKIETHKLTFRENAKAKTDHGAEIVYKSPVVSGDTSPRHLSNVSSTGSIDMVDSPQLATLADEVSASLAKQGLPKKKRKV

SEQ ID NO: 9 - protein, 509 aa, artificial
  fusion protein
HIDDDDKIIGGHEAKPHSRPYMAFLMIWDQKSLKRCGGFLIRDDFVLTAAHCWGSSINVTLGAHNIKEQEPTQQFIPVKRAIPHPAYNPKNFSNDIMLLQLERKAKRTRAVQPLRLPSNKAQVKPGQTCSVAGWGQTAPLGKHSHTLQEVKMTVQEDRKCESDLRHYYDSTIELCVGDPEIKKTSFKGDSGGPLVCNKVAQGIVSYGRNNGMPPRACTKVSSFVHWIKKTMKRYGSKLAEHEGDAAQPAMAQVQLVESGGGVVQPGRSLRLSCSSSGFIFSDNYMYWVRQAPGKGLEWVATISDGGSYTYYPDSVKGRFTISRDNSKNTLFLQMDSLRPEDTGVYFCARGYYRYEGAMDYWGQGTPVTVSSGGGGSGGGGSGGGGSDIQLTQSPSSLSASVGDRVTITCKSSQSVLYSSNQKNYLAWYQQKPGKAPKLLIYWASTRESGVPSRFSGSGSGTDFTFTISSLQPEDIATYYCHQYLSSWTFGQGTKLEIKAAAGPHHHHHH

SEQ ID NO: 10 - protein, 505 aa, artificial
  fusion protein
HIDDDDKIIGGHEAKPHSRPYMAFLMIWDQKSLKRCGGFLIRDDFVLTAAHCWGSSINVTLGAHNIKEQEPTQQFIPVKRAIPHPAYNPKNFSNDIMLLQLERKAKRTRAVQPLRLPSNKAQVKPGQTCSVAGWGQTAPLGKHSHTLQEVKMTVQEDRKCESDLRHYYDSTIELCVGDPEIKKTSFKGDSGGPLVCNKVAQGIVSYGKNNGMPPRACTKVSSFVHWIKKTMKRYAEHEGDAAQPAMAQVQLVESGGGVVQPGRSLRLSCSSSGFIFSDNYMYWVRQAPGKGLEWVATISDGGSYTYYPDSVKGRFTISRDNSKNTLFLQMDSLRPEDTGVYFCARGYYRYEGAMDYWGQGTPVTVSSGGGGSGGGGSGGGGSDIQLTQSPSSLSASVGDRVTITCKSSQSVLYSSNQKNYLAWYQQKPGKAPKLLIYWASTRESGVPSRFSGSGSGTDFTFTISSLQPEDIATYYCHQYLSSWTFGQGTKLEIKAAAGPHHHHHH

SEQ ID NO: 11 - protein, 510 aa, artificial
  fusion protein
HIDDDDKIIGGREVIPHSRPYMASLQRNGSHLCGGVLVHPKWVLTAAHCLAQRMAQLRLVLGLHTLDSPGLTFHIKAAIQHPRYKPVPALENDLALLQLDGKVKPSRTIRPLALPSKRQVVAAGTRCSMAGWGLTHQGGRLSRVLRELDLQVLDTRMCNNSRFWNGSLSPSMVCLAADSKDQAPCKGDSGGPLVCGKGRVLAGVLSFSSRVCTDIFKPPVATAVAPYVSWIRKVTGRSAAEHEGDAAQPAMAQVQLVESGGGVVQPGRSLRLSCSSSGFIFSDNYMYWVRQAPGKGLEWVATISDGGSYTYYPDSVKGRFTISRDNSKNTLFLQMDSLRPEDTGVYFCARGYYRYEGAMDYWGQGTPVTVSSGGGGSGGGGSGGGGSDIQLTQSPSSLSASVGDRVTITCKSSQSVLYSSNQKNYLAWYQQKPGKAPKLLIYWASTRESGVPSRFSGSGSGTDFTFTISSLQPEDIATYYCHQYLSSWTFGQGTKLEIKAAAGPHHHHHH